(19) United States
(12) Patent Application Publication
(10) Pub. No.: US 2024/0188921 A1
(43) Pub. Date: Jun. 13, 2024

(54) Method for determining respiratory phases in an acoustic signal, computer program product, storage medium and corresponding device

(71) Applicants: CONTROLE INSTRUMENTATION ET DIAGNOSTIC ELECTRONIQUES-CIDELEC, SAINTE-GEMMES-SUR-LOIRE (FR); ECOLE SUPERIEURE ELECTRONIQUE DE L'OUEST, ANGERS (FR)

(72) Inventors: Nathalie Freycenon, BRISSAC-LOIRE-AUBANCE (FR); Roberto Longo, ANGERS (FR); Sébastien Menigot, ANGERS (FR)

(21) Appl. No.: 18/554,100

(22) PCT Filed: Apr. 4, 2022
(86) PCT No.: PCT/EP2022/058849

§ 371 Date: Oct. 5, 2023

(30) Foreign Application Priority Data
Apr. 6, 2021 (FR) FR2103511

(51) Int. Cl. A61B 7/00; A61B 5/00; G06N 20/00

(52) U.S. Cl. CPC A61B 7/003; A61B 5/725; G06N 20/00

(57) ABSTRACT

A method for determining respiratory phases in an acoustic signal which represents an individual's respiratory activity. The respiratory activity includes inhalation and exhalation phases. The method includes: detecting pause intervals in the acoustic signal; and determining time intervals corresponding to inhalation phases each between a long pause and a short pause, and time intervals corresponding to exhalation phases each between a short pause and a long pause.

### TECHNICAL FIELD

The invention is part of the field of processing acoustic signals, in particular tracheal or pulmonary sounds captured from an individual.

More specifically, the invention relates to a technique for recognising the respiratory phases of an individual from an acoustic signal representative of the respiratory activity of this individual.

The invention applies in particular, but not exclusively, in the field of acoustic instrumentation dedicated to the recording and monitoring of the respiratory activity of individuals, for example for the study of respiratory pathologies or respiratory disorders during sleep.

### TECHNOLOGICAL BACKGROUND

In the remainder of this document, focus is more particularly made to describe the problem existing in the context of the study of the respiratory activity in patients suffering from “sleep apnoea syndrome” (known as SAS syndrome). The invention is of course not limited to this particular context of application, but is of interest for any technique for recognising respiratory phases having to deal with a close or similar problem.

The sleep apnoea syndrome is manifested by repeated and uncontrolled interruptions in breathing during sleep. They cause non-conscious micro-arousals that can harm the quality of sleep. This can therefore result in daytime drowsiness, difficulties with concentration or memory, or even cardiovascular complications, possible sources of corollary accidents such as road or work accidents. It is therefore important to have medical equipment capable of detecting this type of pathology, in particular for screening and treatment purposes.

The diagnosis of sleep apnoea syndrome is mainly based on the exploration of the individual's ventilation in order to observe movements of breathing and the evolution of the upper airway during sleep. Apnoeas and hypopneas are investigated by analysing the amplitude of acoustic signals obtained for example during ventilatory polygraphy or polysomnography. It is generally recommended to use a certain number of measuring instruments to measure the individual's respiratory behaviour during sleep, such as nasal cannula, thoraco-abdominal straps, pulse oximeter, etc. An analysis software analyses the signals recorded by the different measuring instruments in order to identify possible pathological events, such as apnoeas and hypopneas. Other instruments, such as tracheal sound sensors, can also be used in a complementary manner to study more specifically certain parameters associated with the inhalation and exhalation phases of the individual's respiratory activity, and allow refining the diagnosis. Indeed, the latest studies carried out on respiratory cycles obtained from tracheal sound signals have allowed highlighting a certain number of pathological indicators whose consideration proves to be relevant in the detection and evaluation of respiratory disorders.

However, even if there are techniques for analysing respiratory cycles from tracheal sounds, such as the non-invasive phase detection technique proposed by ZK Moussavi et Al. in the scientific article “Computerised acoustical respiratory phase detection without airflow measurement” (March 2000), these lack accuracy. The respiratory activity of an individual (typically composed of successive respiratory cycles, each respiratory cycle comprising an inhalation phase and an exhalation phase) can be observed from the following three representations: a temporal representation, a frequency representation and a frequency-intensity representation over time (“three-dimensional” representation). However, it proves that it is often difficult to accurately distinguish the inhalation phase from the exhalation phase of a respiratory cycle in the frequency or time domain of a tracheal sound signal, in particular when the captured signal is located below a certain sound threshold (case of an individual at rest for example). Moreover, such known techniques only provide a limited amount of relatively imprecise information on the patient's respiratory activity.

Moreover, certain respiratory profiles prove to be particularly complex to process, for example in the case of snoring or in the case of apnoea during which the reduction in the acoustic signal is accompanied by noises linked to the individual's efforts to enter or exit air via the respiratory tract. Other noises, such as swallowing, coughing, throat clearing, or even changes in the individual's position during his sleep complicate the recognition of the respiratory phases.

There is therefore a real need to provide a technique which allows an effective recognition of the respiratory phases of an individual in a tracheal sound signal, and more generally in an acoustic signal representative of a respiratory activity. It would also be particularly interesting to provide such a technique which is minimally invasive and easy to implement.

### DISCLOSURE OF THE INVENTION

In a particular embodiment of the invention, a method is proposed for determining respiratory phases in an acoustic signal representative of a respiratory activity of an individual, the respiratory activity being defined by respiratory cycles each comprising an inhalation phase and an exhalation phase, the method is remarkable in that it comprises the following steps:
detecting pause intervals in the acoustic signal;
determining activity intervals corresponding to inhalation phases and activity intervals corresponding to exhalation phases by comparison, for each time interval, of the duration of the pause interval which precedes said time interval with the duration of the pause interval which follows said time interval.

Thus, the principle of the invention is based on a search in the acoustic signal for pauses of the signal to deduce the inhalation and exhalation phases therefrom thanks to their duration and their position in the signal. This new approach is clever because, rather than directly seeking to process the inhalation and exhalation phases as in the state of the art, the present invention focuses first on recognising pauses in the signal, these being easier to recognise in an acoustic signal. Thus, the invention offers a technique for recognising respiratory phases which is more effective than state-of-the-art techniques. Moreover, being able to determine the inhalation and exhalation phases using only an acoustic signal offers prospects for simplified examination of medical equipment.

According to a particular aspect, the method implements a mechanism for distinguishing among the pause intervals, between a first type of pause interval, called short pause, of duration less than a second type of pause interval, called long pause.

According to a particular feature, the acoustic signal belongs to the group comprising: a tracheal acoustic signal or a pulmonary acoustic signal captured from an individual.

According to a particular implementation of the method, if at the end of the detection step at least one succession of two pause intervals which are separated in the signal by a time interval, called aberrant interval, of duration less than a predetermined duration threshold is identified, the method comprises a step of replacing, for each detected succession, the set consisting of the two pause intervals and said aberrant interval by a new pause interval. The mechanism for recognising the inhalation and exhalation phases according to the invention is thus optimised. Indeed, the predetermined threshold is dimensioned such that when a point defect appears in the signal between two pause intervals, this is considered as an integral part, with the two pause intervals, of the same and single pause. This allows eliminating the interference noise contained in the signal which would disrupt the recognition of the respiratory phases.

According to a particular feature, if at the end of the determination step at least one time interval, called indeterminate interval, meeting a predetermined inconsistency criterion, is identified: —processing the signal portion corresponding to said indeterminate interval, by means of a machine learning method; —associating said at least one indeterminate interval with an inhalation or exhalation phase depending on the results of the processing step. The mechanism for recognising the inhalation and exhalation phases is thus further improved according to the invention.

According to a particular aspect of the invention, the step of detecting pause intervals is based on a processing belonging to the group comprising a frequency processing of the acoustic signal, an energy processing of the acoustic signal. Indeed, it is by observing the energy and time-frequency representation spaces of sound signals that has been considered, surprisingly, that the respiratory pauses are more easily recognisable in these representation spaces than the inhalation and exhalation phases themselves. The pause recognition mechanism according to the invention can be implemented either by frequency processing of the signal, or by energy processing of the signal, or by simultaneous frequency and energy processing of the signal, thus making the method even more reliable.

According to a particular implementation, the frequency processing comprises the following steps:
applying a high-pass anti-noise filter to said signal to remove a first predefined frequency range from said signal and obtain a filtered signal as a function of time;
segmenting said filtered signal to obtain a plurality of samples of said filtered signal and, for each sample, estimating the average power spectral density of said sample so as to obtain a frequency signal representing the centroid frequency as a function of time;
applying a sliding time window of predetermined duration to said frequency signal and, for each window:
detecting a frequency maximum of the frequency signal in said window,
determining a frequency threshold depending on the frequency maximum detected in said window;
shaping, from said frequency signal, a first transient logic signal of logic levels defined depending on the frequency thresholds determined for said frequency signal, the pause intervals being detected depending on the first transient logic signal.

According to a particular implementation, the energy processing comprises the steps:
applying a high-pass anti-noise filter and a low-pass filter to remove respectively first and second predefined frequency ranges from said signal and obtain a filtered signal as a function of time;
applying a first sliding time window of predetermined duration to said filtered signal and, for each time window, estimating the acoustic intensity of said filtered signal by applying a root mean square envelope in said window, so as to transform said filtered signal into an energy signal as a function of time;
applying a second sliding time window of duration determined by autocorrelation to said energy signal and, for each window:
detecting a minimum intensity of the energy signal in said window,
determining an intensity threshold as a function of the minimum intensity detected in said window;
shaping, from said energy signal, a second transient logic signal of logic levels defined depending on the intensity thresholds determined for said energy signal, the pause intervals being detected depending on the second transient logic signal.

According to a particularly advantageous aspect of the invention, the method comprises a step of shaping, by processing said first and second transient logic signals by an OR-Inclusive logic function, a final logic signal whose high logic levels correspond to said detected pause intervals. Since the frequency and energy treatments can be complementary, taking these two processings into account can allow detecting pauses which would not have been detected by the implementation of only one of the two processings.

According to a particular feature, the method comprises a step consisting in, if at the end of the shaping step, at least one succession of two pause intervals which are separated by an aberrant interval detected in said final logic signal of duration less than the predetermined duration threshold is identified: replacing, for each succession detected in said final logic signal, the set consisting of the two pause intervals and said aberrant interval by a new pause interval. This facilitates distinguishing between the long pauses and short pauses, and in fact allows a more reliable recognition of respiratory phases.

In another embodiment of the invention, a computer program product is proposed, which comprises program code instructions for implementing the aforementioned method (in any one of the different embodiments thereof), when said program is executed on a computer.

In another embodiment of the invention, a computer-readable and non-transitory storage medium is proposed, storing a computer program product comprising a set of instructions executable by a computer to implement the aforementioned method (in any one of the different embodiments thereof).

In another embodiment of the invention, a device is proposed for determining respiratory phases in an acoustic signal representative of a respiratory activity of an individual, the respiratory activity being defined by respiratory cycles each comprising an inhalation phase and an exhalation phase, the device being characterised in that it comprises: —means for detecting pause intervals in the acoustic signal; —means for determining activity intervals corresponding to inhalation phases and activity intervals corresponding to exhalation phases by comparison of the duration of the pause intervals

Advantageously, the device comprises means for implementing the steps which it performs in the obtaining method as previously described, in any one of the different embodiments thereof.

### LIST OF FIGURES

Other features and advantages of the invention will appear on reading the following description, given by way of indicative and non-limiting example, and the appended drawings, in which:

FIG. 1 shows a flowchart of a particular embodiment of the method according to the invention;

FIG. 2 illustrates in a simplified manner the principle of recording a respiratory activity of an individual by means of a tracheal sound sensor;

FIG. 3 shows a flowchart illustrating a particular implementation of a frequency processing and an energy processing of the tracheal acoustic signal in accordance with the invention;

FIG. 4 is a temporal graphical representation illustrating the evolution of the intensity of a tracheal acoustic signal recorded on an individual;

FIG. 5 is a temporal graphical representation illustrating the principle of frequency processing of the acoustic signal according to the invention;

FIG. 6 is a temporal graphical representation of the chronogram type highlighting the respiratory pauses contained in an acoustic signal by frequency processing;

FIG. 7 is a temporal graphical representation illustrating the principle of energy processing of the acoustic signal according to the invention;

FIG. 8 is a temporal graphical representation of the chronogram type highlighting the respiratory pauses detected in the acoustic signal by energy processing;

FIG. 9 represents a final chronogram highlighting the respiratory pauses detected in the acoustic signal from frequency and energy processing;

FIG. 10 represents a final chronogram illustrating the principle of determining the inhalation and exhalation phases depending on the pauses detected in the acoustic signal;

FIG. 11 represents the simplified structure of a device implementing the method according to a particular embodiment of the invention.

### DETAILED DESCRIPTION OF THE INVENTION

In all figures of the present document, identical elements and steps are designated by the same reference numerals.

In the remainder of the description, we consider an example of implementation of the invention in the context of a tracheal acoustic signal, that is to say an acoustic signal obtained on the trachea of an individual to carry out an examination of respiratory activity of said individual during sleep (for example to diagnose a sleep apnoea syndrome). The invention is of course not limited to this particular context, and can be applied to any type of acoustic signals representative of a respiratory activity of a living being.

FIG. 1 represents, generically, a flowchart of a particular embodiment of the method according to the invention. This flowchart illustrates the main steps of implementing the method, denoted S1 to S4. These steps are implemented by a device (the principle of which is described later in relation to FIG. 11) and aim at determining the inhalation and exhalation phases of an individual's respiratory activity.

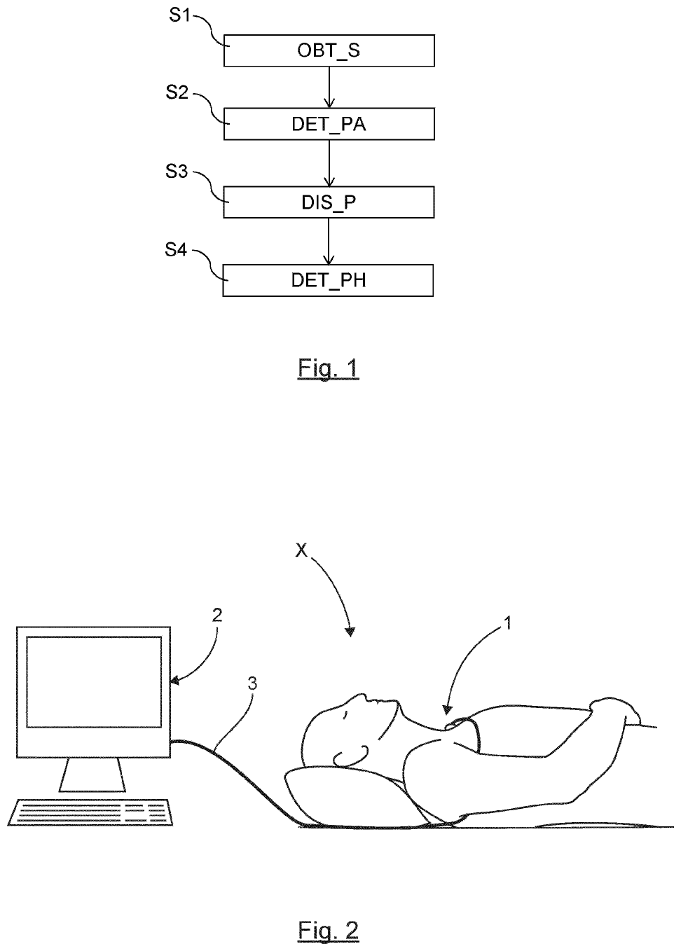

FIG. 2 shows an individual X, in a lying position, undergoing a respiratory examination, for example a ventilatory polygraphy. The individual X has, at his trachea, an acoustic sensor, held by a medical adhesive. Such a sensor 1 is configured to capture sound pressure waves from the trachea of the individual X during his sleep. These sound pressure waves are transformed by the sensor into analogue electrical signals, the latter then being converted into digital signals before being transmitted to a processing unit 2. The processing unit 2 is connected to the sensor 1 via an electrical connection 3 (or via a short-range wireless link for example). In order to allow a processing over a sufficiently large frequency range, the sensor 1 is configured so as to have a bandwidth typically extending between 10 Hz and 10 kHz. Of course, this frequency range is given by way of illustrative example and other ranges can be considered without departing from the scope of the invention depending on the intended application or the practiced examination. The processing unit 2, for its part, is configured to receive, store and process the signals transmitted by the sensor 1. It has the program code instructions allowing the implementation of the method of the present invention. A man-machine interface connected to the processing unit allows the user (the medical staff in this case) to follow the evolution of the respiratory activity of the individual X and to execute the method of the present invention for analysis and diagnosis purposes.

In the remainder of the document, the term “acoustic signal” means the electrical signals representative of the respiratory activity of the individual X which have been collected and recorded by the processing unit 2 during the examination.

The individual's respiratory activity is typically composed of respiratory cycles, each cycle comprising an inhalation phase and an exhalation phase which produce sound pressure waves within the individual. The inhalation phase corresponds to a filling of the lungs during which the diaphragm and the intercostal muscles are contracted. The exhalation phase corresponds to an emptying of the lungs during which the thoracic muscles are relaxed.

The method is initialised by activating the recording of the respiratory activity of the individual X or after a predetermined recording duration.

In step S1 (referenced “OBT_S”), the device acquires the tracheal acoustic signal of the individual X. An example of a tracheal acoustic signal (referenced “SAT”) is represented in FIG. 4.

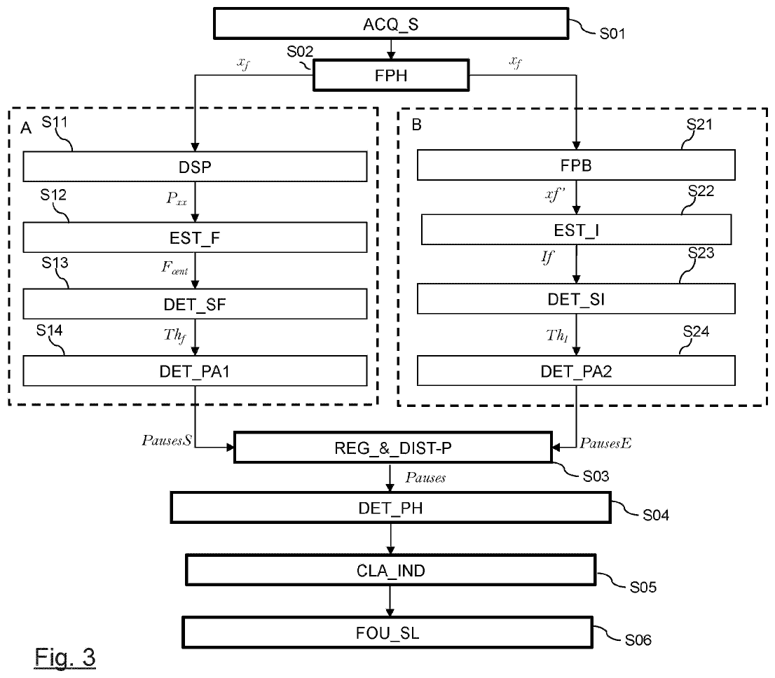

In step S2 (referenced “DET_PA”), the device proceeds, by signal analysis, to a detection of the pause intervals contained in the SAT signal. This detection step is based on the implementation, by the device, of a frequency processing of the SAT signal and/or an energy processing of the SAT signal. The principle of these two processings is described in detail below in relation to the blocks A and B of the flowchart in FIG. 3. The detected pause intervals are stored in a local table of the device.

The term “pause interval” means a time interval during which the corresponding signal portion is representative of a respiratory pause, in other words of an absence of respiratory activity. This absence of respiratory activity characteristic of a cycle of conventional respiratory activity is easily identified in the signal both in the frequency domain and in the energy domain.

In step S3 (referenced “DIS_P”), the device performs a distinction, among the pause intervals detected in step S2, between a first type of pause interval, called “short pause” and a second type of pause interval, called “long pause”, depending on the estimated duration for each of the pause intervals. It is considered here that a “short pause” type interval is of a duration less than a “long pause” type interval.

According to a particular implementation, after having estimated the duration of the detected pause intervals, the device carries out a comparison of the duration of the pause intervals which follow each other in pairs over time to determine which one is the short pause and which one is the long pause.

Complementarily or alternatively, the device compares the duration of each of the pause intervals depending on a predetermined threshold. Such a threshold consists of a duration chosen to differentiate the long pauses from the short pauses among the detected pause intervals. For example, a pause interval is considered to correspond to a long pause if the estimated duration for this interval is greater than said threshold. Otherwise, it corresponds to a short pause if the estimated duration for this interval is less than said threshold.

In step S4 (referenced “DET_PH”), the device determines the time intervals of the SAT signal corresponding to the inhalation and exhalation phases taking into account the following scheduling rule: a time interval corresponds to an inhalation phase if it is comprised between a long pause and a short pause, and an exhalation phase if it is comprised between a short pause and a long pause. Indeed, in a respiratory cycle in a normal resting situation, the inhalation phase is followed by an inhalation pause (or short pause), which is followed by an exhalation phase, itself followed by an exhalation pause (or long pause), the short pause being of a duration less than the long pause. In other words, the principle is based on the comparison, for each time interval comprised between two successive pauses, of the pause which precedes and the pause which follows said interval. An inhalation is followed by a pause shorter than the one preceding it, an exhalation is followed by a pause longer than the one preceding it. It is based on this observation that the aforementioned scheduling rule was established for the present invention.

Thus, at the end of step S4 of the method, the device has a segmentation of the SAT signal into time intervals each associated with one of the following three categories: an inhalation phase, an exhalation phase, a pause. This information can be provided in the form of a transient logic signal representative of the respiratory activity of the individual X, the logic states of which correspond to the respiratory phases and pauses of said individual. The device communicates the logic signal representative of the respiratory activity of the individual X to the man-machine interface, for display and diagnosis purposes by the medical staff.

Thus, the general principle of the method is based on searching in a tracheal acoustic signal for pauses in the respiratory activity of an individual to deduce the inhalation and exhalation phases contained in this signal. This respiratory phase recognition technique is particularly effective because it focuses on priority processing of signal portions corresponding to respiratory pauses, easily recognisable in the frequency and energy domains. It is indeed by observing the temporal evolution of tracheal acoustic signals in the frequency and energy domains that the inventors of the present noticed that the pauses are more easily identified than the inhalation and exhalation phases as such. By offering the possibility of determining the inhalation and exhalation phases solely using tracheal sounds captured from an individual, the method of the invention thus opens up prospects for simplified and less invasive examination, further requiring lighter diagnosis medical equipment.

In relation to FIG. 3, a particular implementation of the method is shown in which steps S2, S3 and S4 of the flowchart described above are more fully detailed.

After recovering the tracheal acoustic signal SAT in step S01 (referenced “ACQ_S”), the device first applies an anti-noise high-pass filter to the signal in step S02 (referenced “FPH”), by example a Butterworth type filter of order 4 having a cutoff frequency equal to 100 Hz. This high-pass filter has the function of removing, from the SAT signal, a predefined frequency range corresponding to the cardiovascular noises emitted by the individual X through his trachea. The signal obtained at the output of the high-pass filter is a signal filtered in the time domain, subsequently called filtered signal ‘xf’.

The observation of the temporal frequency-energy representation revealed that it is on this type of representation that the pauses in respiratory activity are easiest to recognise. Two physical magnitudes distinguish the pauses of the respiratory phases (inhalation/exhalation): the frequency content and the energy. Indeed; it appears that the energy of the tracheal acoustic signal is minimal during the pauses since it corresponds to an absence of breathing and that the spectrum of the signal corresponding to a pause is close to that of white noise in the frequency band comprised between 100 and 2000 Hz.

Two particular techniques are proposed for processing the filtered signal ‘xf’: a frequency processing (represented by the dotted block A) and an energy processing (represented by the dotted block B). The second technique is subsequently described.

### Frequency Processing of the Signal

In step S11, the device carries out a segmentation of the filtered signal xf to obtain a plurality of samples of this signal. For example, the filtered signal xf is divided into segments of 200 samples each corresponding to 50 ms. Each segment, denoted ‘l’, is further divided into three sub-segments which overlap at 50%. Then, the device estimates the power spectral density (also known as “PSD”) of each segment l so as to transform the filtered signal xf into a frequency signal, denoted ‘F_{cent}’ in the time domain.

The estimated PSD of the segment l, denoted ‘Pₓₓ[k,l]’, is equal to the mean of the PSD of the three sub-segments composing the segment l. The index k represents the index of the frequency ‘Fₖ’ such that Fₖ=k. Fₛ/N_{fft}, with the frequency Fₛ equal to 4 kHz, N_{fft} the number of points of the Fourier transform (assuming that N_{fft} is an even number) and k an integer comprised in the interval [0−N_{fft}/2]. For each segment I of the signal xf, the device estimates in step S12 (“EST_F”) the centroid frequency F_{cent}[l] by means of the following relationship:

$${F}_{cent} [ l ] = \frac{{\sum }_{k = 0}^{{N}_{fft} / 2} {F}_{k} {P}_{xx} [ k , l ]}{{\sum }_{k = 0}^{{N}_{fft} / 2} {P}_{xx} [ k , l ]}$$

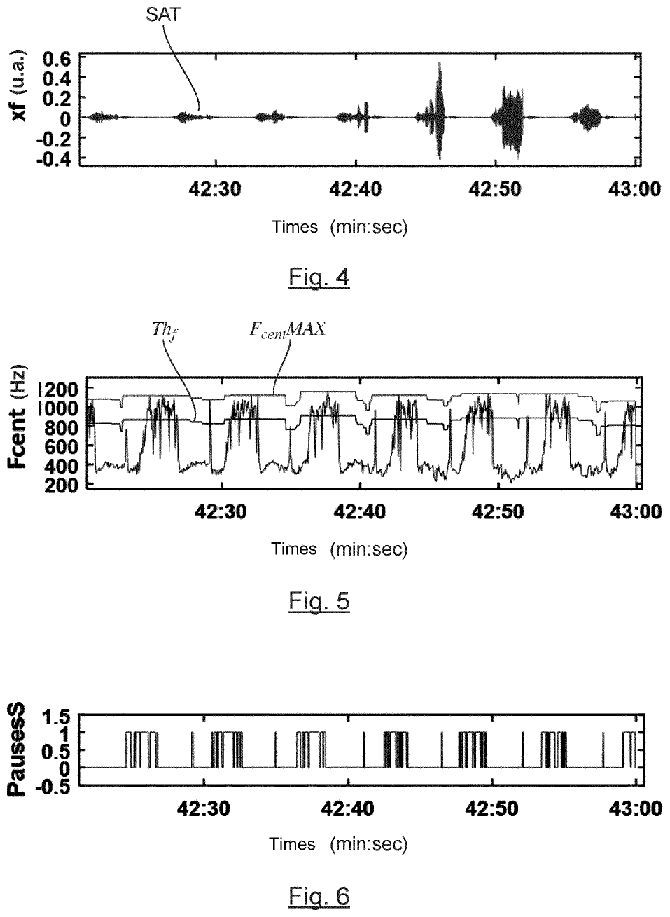

Step S13 (“DET_SF”) consists in determining a variable frequency threshold, denoted Th_{f}, which will be used in step S14 to identify the pause intervals contained in the acoustic signal. To do this, the device defines a sliding time window of predefined size substantially corresponding to the average duration of a respiratory cycle, then applies this sliding window to the frequency signal F_{cent} to determine the frequency threshold whose value is a function of the frequency maximum of the frequency signal F_{cent} in the considered sliding window. By way of example, as illustrated in FIG. 5, a time window of predefined size corresponding to 80 samples (i.e. a window of duration substantially equal to 4 seconds) is applied to the frequency signal F_{cent}. For each applied window, the device determines the maximum value of the frequency of the frequency signal F_{cent} of this window (denoted F_{centMAX} in the figure), and subtracts a predefined frequency value ΔF from this maximum value to deduce the value of the frequency threshold Th_{f} for this window. The predefined frequency value ΔF is typically comprised between 100 Hz and 300 Hz.

Thus, rather than defining a frequency threshold of predetermined value, the device according to the invention proposes a calculation of a variable threshold coming as close as possible to the shape of the frequency signal, which allows providing a method with increased reliability.

It should be noted that the centroid frequency is close to 1000 Hz during the pauses, which corresponds to the Nyquist frequency divided by two. This result is consistent with the hypothesis of white noise in a frequency band comprised between 100 and 2000 Hz.

In step S14 (“DET_PA1”), the device proceeds to shape, from the frequency signal F_{cent}, a transient logic signal PauseS whose logic levels are depending on the frequency threshold values Th_{f} determined in step S13. An example of a logic signal PauseS, resulting from a frequency processing, is represented in FIG. 6. This signal PauseS is representative of the pauses contained in the SAT signal. Thus, for a given point of the frequency signal F_{cent}, if the frequency value associated with this point is greater than the frequency threshold value Th_{f} determined for this point, then the logic level of the signal PauseS takes the value 1, which means that a pause interval is detected. Conversely, if the frequency value associated with this point is equal to or less than the frequency threshold value Th_{f} determined for this point, then the logic level of the signal PauseS takes the value 0, which means that no pause interval is detected. The detected pause intervals are then stored in the device's local table. The frequency processing ends at the end of this step S14.

### Energy Processing of the Signal

The device first applies, in step S21, a low-pass filter to the filtered signal xf (referenced “FPB”) in order to remove the frequencies for which the tracheal sound intensity level is low. The application of a Butterworth type filter of order 6 having a cutoff frequency equal to 1500 Hz, for example, leads to a filtered signal xf′ whose frequency range is comprised between 100 and 1500 Hz, corresponding to the most energetic frequency band of the tracheal sounds.

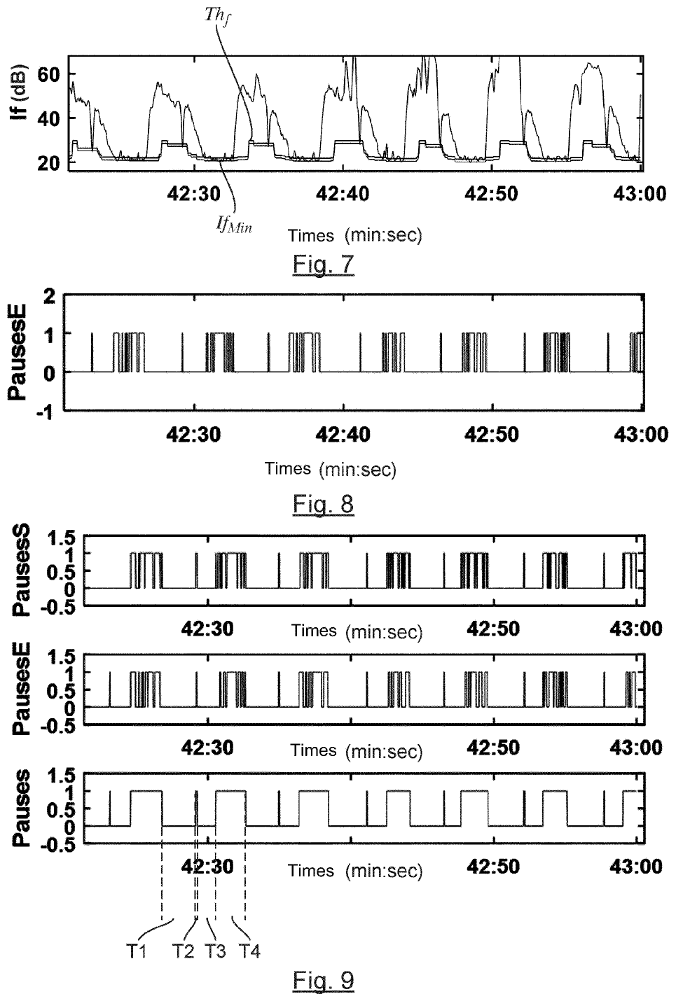

In step S22 (“EST_I”), the device defines a first sliding time window of predefined size, then applies this first window to the filtered signal xf′ in order to estimate the acoustic intensity of this filtered signal. By way of example, as illustrated in FIG. 7, a root mean square envelope or RMS envelope or even square root envelope of the mean square, is calculated from the filtered signal xf′ by using a sliding time window of size equal to 125 samples for example (i.e. a window of duration substantially equal to 0.031 s), so as to obtain an energy signal, denoted ‘I_{f}’, evolving in the time domain (the acoustic intensity level of the filtered signal xf′ being proportional to the magnitude of the RMS envelope calculated from this signal).

Step S23 (“DET_SI”) consists in determining the variable intensity threshold, denoted Thₜ, which will be used in step S24 to identify the pause intervals contained in the acoustic signal.

To do this, the device determines a second sliding time window, but this time of variable size W depending on the respiratory period P_{R}. The respiratory period P_{R} is itself determined by means of an autocorrelation function, denoted r_{I}_{_{f}}, applied to the energy signal I_{f} using the following equation:

$${r}_{{I}_{f}} [ l ] = \frac{1}{N} {\sum }_{k = 0}^{N - 1} {I}_{f} [ k ] {I}_{f} [ k + l ]$$

with:
k, an integer comprised between 0 and N−1,
N, the number of points of the autocorrelation window, with N=240,000 (corresponding to 60 seconds at the sampling frequency Fₛ=4 kHz),
l, an integer comprised between (−N+1) and (N−1).

For l≥2000 (i.e. ½ s), the respiratory period P_{R} is deduced from the location of the maximum of the function r_{I}_{_{f}}[l]. Then, it is considered that the size W of the second sliding time window represents a fraction, less than 0.5, of the respiratory period P_{R} (for example 0.4×P_{R}) in order to take into account the shortest duration that an inhalation phase may have. The search at ½ second is started because the autocorrelation function is maximum when l=0.

Then, the device applies the second sliding windows determined above to the energy signal I_{f} in order to identify the minimum value of the sound intensity of the energy signal I_{f} (denoted I_{Min}) appearing for each second sliding window determined above. To do this, the device searches, for a given moment of the signal, the minimum sound intensity 0.2×P_{R} before and 0.2×P_{R} after said moment. Then, for each considered sliding window, a predefined intensity value Δl is added to the minimum sound intensity value determined for said considered window, so as to deduce the intensity threshold value to be taken into account for said considered window. The predefined intensity Al is typically comprised between 1 and 2 dB.

Thus, the device of the invention proposes a variable threshold calculation getting as close as possible to the shape of the energy signal, which allows providing a method with increased reliability.

In step S24 (“DET_PA2”), the device proceeds to shape, from the energy signal I_{f}, a transient logic signal PauseE whose logic levels depend on the intensity threshold values Th_{I} determined in step S13. An example of logic signal PauseE is represented in FIG. 8. As for the logic signal PauseS, this signal PauseE is representative of the pauses contained in the SAT signal. Thus, for a given point of the signal I_{f}, if the sound intensity level associated with this point is lower than the intensity threshold determined for this point, then the logic level of the signal PauseE takes the value 1, meaning that a pause interval is detected. Conversely, if the intensity level associated with this point is greater than or equal to the intensity threshold determined for this point, then the logic level of the signal PauseE takes the value 0, meaning that no pause interval is detected.

The pause intervals thus detected are then stored in the local table of the device. The frequency processing ends at the end of this step S24.

At the end of processing steps S14 and S24, the device can decide to return the chronograms of the current logic signals PauseE and PauseS to the user via the man-machine interface, in order to highlight the respiratory pauses contained in the SAT signal via the frequency processing and the energy processing.

In step S03 (“REG_&_DIST-P”), the device has a first set of pause intervals resulting from the frequency processing (chronogram of the logic signal PauseS) and a second set of pause intervals resulting from the energy processing (chronogram of the logic signal PauseE). The device will then process the logic signals PauseS and PauseE by an ‘OR-Inclusive’ logic function so as to obtain a final logic signal ‘Pause’ whose high logic levels correspond to the detected pause intervals. As illustrated in FIG. 9, in the case of the presence of a logic level of value 1 for at least one of the logic signals PauseS and PauseE, the logic level of the final signal takes the value 1 (meaning that a pause interval is detected), otherwise the logic level of the final signal takes the value 0 (meaning that no pause interval is detected). The device thus groups together the pause intervals from the two chronograms PauseS and PauseE.

Thus, as previously described, the embodiment described here is based on a simultaneous implementation of the two signal processing operations. Such an embodiment makes the method even more reliable and robust. It is entirely possible, as an alternative, to implement only one of the two aforementioned processing operations to identify the pause intervals in the acoustic signal, for example to gain processing speed or even because one of the two processings is more adapted to the nature of the studied acoustic signal.

The device first checks whether successions of pause intervals separated by a time interval— called aberrant interval— of a duration which is less than a predetermined threshold are present in the logic signal obtained at the end of step S03. The value of this predetermined threshold is chosen to be of very short duration, that is to say a duration typically less than ¼ s. Thus, a time interval comprised between two pauses of duration less than the predetermined threshold is considered by the device as an aberration of the signal and must be part of a single pause interval. In the case of positive check for two given successive pauses, the time interval bringing together the two pauses of said succession and the aberrant interval is replaced, in the final logic signal, by a new pause interval (of logic level 1). In the case of a negative check, no replacement is made.

In the following step S04 (“DET_PH”), the device determines the inhalation and exhalation phases of the acoustic signal by comparing, for each remaining time interval, the duration of the preceding pause relative to the duration which follows said interval. The device considers that a time interval of the low logic level signal corresponds to:
an inhalation phase if it is comprised between a long pause and a short pause; or
an exhalation phase if it is comprised between a short pause and a long pause.

Indeed, it is recalled that in a typical respiratory cycle, an inhalation is preceded by a long pause and followed by a short pause and an exhalation is preceded by a short pause and followed by a long pause.

A simple comparison of the relative duration of the pauses preceding and following each time interval is thus carried out by the device. It is therefore not necessarily useful to measure the duration then to allocate a duration state depending on this measurement to the detected pauses. Alternatively, it is possible to configure the device so that it calculates the absolute value of the duration of the detected pauses, then to compare the calculated values to deduce the exhalation and inhalation phases of the signal.

This information is stored in a local table of the device.

As illustrated in FIG. 9, in step S04, the device knows, by comparison of the durations, that the pause T2 of the signal PauseS is a short pause and that the pause T4 is a long type pause. As an exhalation phase is preceded by a short pause and followed by a long pause, it deduces therefrom, in step S04, that the respiratory activity interval T3 corresponds to an exhalation phase.

Step S05 (“CLAJND”) consists in further improving the determination algorithm according to the invention by checking whether, for certain time intervals, the respiratory phase to which they are allocated would not be inconsistent with a conventional respiratory cycle. This step is carried out depending on one or more predetermined inconsistency criteria relating to the human respiratory cycle. A succession of at least two inhalation or exhalation phases constitutes an example of an inconsistency criterion applicable to the present invention. The cycles that do not meet the consistency criteria are then classified as indeterminate. The person skilled in the art is able to adapt the list of possible inconsistency criteria and to possibly combine them depending on the weight he wishes to give to this step of the algorithm. In the case of a positive check for a given interval, the latter is considered an “indeterminate interval”. For each indeterminate interval of the signal, the device processes the signal portion corresponding to said indeterminate interval by means of a supervised machine learning method, such as for example the method known as “Random forest” based on trees of random decision. This method is adapted to each patient by carrying out the learning phase using the supposedly well-classified cycles of said patient. The features are extracted from the filtered signal xf, for example the duration, energy, power, shape, spectral content, or any other feature that the person skilled in the art could determine.

In a complementary or alternative manner, a simple processing of the indeterminate intervals based on the coherence of the successions of the respiratory phases and the known durations of the inhalation and exhalation phases, can be implemented prior to or simultaneously with the learning method for allow quickly and easily reclassifying all or part of the indeterminate intervals.

Thanks to this step, the mechanism for recognising the inhalation and exhalation phases is further refined when certain determined phases meet said inconsistency criterion.

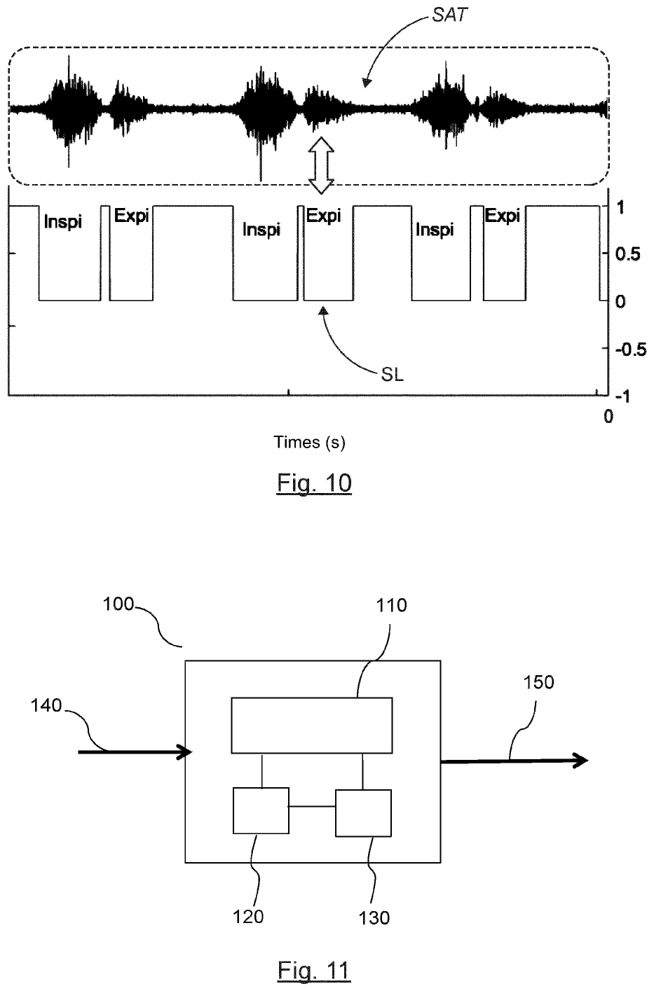

Finally, in step S06 (“FOU_SL”), the device provides, via the man-machine interface, the information relating to the respiratory phases which are previously determined in the form of a transient logic signal capable of being interpreted by qualified medical staff. As illustrated by way of example in the chronogram in FIG. 10, such a logic signal SL has a low logic level (of value equal to 0) when it is an inhalation phase (denoted “Inspi” in the figure) or an exhalation phase (denoted “Expi”), and a high logic level (of value equal to 1) when it is a pause. The parameters associated with the inhalation and exhalation phases of the respiratory activity of the individual X can be deduced from this logic signal SL for diagnosis purposes.

FIG. 11 shows the simplified structure of a device 100 implementing the determination method according to the invention (for example the particular embodiment described above in relation to FIGS. 1 to 10). This device comprises a random access memory 130 (for example a RAM memory), a processing unit 110, equipped for example with a processor, and driven by a computer program stored in a read-only memory 120 (for example a ROM memory or hard disk). At initialisation, the code instructions of the computer program are for example loaded into the random-access memory 130 before being executed by the processor of the processing unit 110. The processing unit 110 receives the tracheal acoustic signal 140 (for example the SAT signal) as input. The processor of the processing unit 110 processes the signal 140 according to the instructions of the computer program and generates a final transient logic signal, highlighting the different respiratory phases of the individual, as output 150. This processing is carried out using means for detecting pause intervals, means for distinguishing long pauses or short pauses and means for determining respiratory phases depending on the detected pauses.

This FIG. 11 illustrates only one particular manner, among several possible manners, of carrying out the different algorithms detailed above, in relation to FIGS. 1 and 3. Indeed, the technique of the invention is carried out indifferently:
on a reprogrammable calculation machine (a PC computer, a DSP processor or a microcontroller) executing a program comprising a sequence of instructions, or
on a dedicated calculation machine (for example a set of logic gates such as an FPGA or an ASIC, or any other hardware module).

In the case where the invention is implemented on a reprogrammable calculation machine, the corresponding program (that is to say the sequence of instructions) could be stored in a removable storage medium (such as for example a floppy disk, a CD-ROM or a DVD-ROM) or not, this storage medium being partially or totally readable by a computer or a processor.

The particular embodiment presented above is based on the processing of a tracheal acoustic signal. Of course, it is entirely possible to apply the method of the invention to an acoustic signal from another organ of the individual, such as a pulmonary acoustic signal for example (to mention only one example of application). More generally, the technique of the invention applies to any type of acoustic signals representative of the respiratory activity of an individual.

1. A method comprising:
receiving an acoustic signal as input to a device and storing the acoustic signal in a non-transitory computer readable medium;
processing the stored acoustic signal by the device to determine respiratory phases in the acoustic signal, the acoustic signal being representative of a respiratory activity of an individual, the respiratory activity being defined by respiratory cycles each comprising an inhalation phase and an exhalation phase, the processing comprising:
detecting pause intervals in the acoustic signal; and
determining time intervals corresponding to inhalation phases and time intervals corresponding to exhalation phases by comparison, for each time interval, of a duration of the pause interval which precedes said time interval with a duration of the pause interval which follows said time interval.

2. The method according to claim 1, comprising, in response to the device identifying, at an end of the detecting, at least one succession of two pause intervals which are separated in the signal by a time interval, called aberrant interval, of duration less than a predetermined duration threshold:
replacing, for each detected succession, a set consisting of the two pause intervals and said aberrant interval by a new pause interval.

3. The method according to claim 1, comprising, in response to the device identifying, at the end of the determining, at least one time interval, called indeterminate interval, meeting a predetermined inconsistency criterion:
processing, for each indeterminate interval, a portion of the acoustic signal corresponding to said indeterminate interval, by using a machine learning method; and
associating said at least one indeterminate interval with an inhalation or exhalation phase depending on results of processing the portion of the acoustic signal.

4. The method according to claim 1, wherein detecting the pause intervals is based on a processing belonging to the group consisting of:
a frequency processing of the acoustic signal;
an energy processing of the acoustic signal.

5. The method according to claim 4, wherein detecting the pause intervals is based on the frequency processing of the acoustic signal, and the frequency processing comprises:
applying a high-pass anti-noise filter to said signal to remove a first predefined frequency range from said signal and obtain a filtered signal as a function of time;
segmenting said filtered signal to obtain a plurality of samples of said filtered signal and, for each sample, estimating an average power spectral density of said sample so as to obtain a frequency signal representing the centroid frequency as a function of time;
applying a sliding time window of predetermined duration to said frequency signal and, for each window:
detecting a frequency maximum of the frequency signal in said window,
determining a frequency threshold depending on the frequency maximum detected in said window;
shaping, from said frequency signal, a first transient logic signal of logic levels defined depending on the frequency thresholds determined for said frequency signal, the pause intervals being detected depending on the first transient logic signal.

6. The method according to claim 4, wherein detecting the pause intervals is based on the energy processing of the acoustic signal, wherein the energy processing comprises:
applying a high-pass anti-noise filter and a low-pass filter to remove respectively first and second predefined frequency ranges from said signal and obtain a filtered signal as a function of time;
applying a first sliding time window of predetermined duration to said filtered signal and, for each time window, estimating the acoustic intensity of said filtered signal by applying a root mean square envelope in said window, so as to transform said filtered signal into an energy signal as a function of time;
applying a second sliding time window of duration determined by autocorrelation to said energy signal and, for each window:
detecting a minimum intensity of the energy signal in said window,
determining an intensity threshold determined as a function of the minimum intensity detected in said window;
shaping, from said energy signal, a second transient logic signal of logic levels defined depending on the intensity thresholds determined for said energy signal, the pause intervals being detected depending on the second transient logic signal.

7. The method according to claim 6, comprising shaping, by processing said first and second transient logic signals by an OR-Inclusive logic function, a final logic signal of high logic levels corresponding to said detected pause intervals.

8. The method according to claim 7, comprising, in response to the device identifying, at the end of the shaping the final logic signal, at least one succession of two pause intervals which are separated by an aberrant interval detected in said final logic signal of duration less than the predetermined duration threshold:
replacing, for each succession detected in said final logic signal, a set consisting of the two pause intervals and said aberrant interval by a new pause interval.

9. (canceled)

10. A computer-readable and non-transitory storage medium storing a computer program product comprising program code instructions for implementing a method, when said program is executed on a processor of a device, the method comprising:
receiving an acoustic signal as input to the device and storing the acoustic signal;
processing the stored acoustic signal by the device to determine respiratory phases in the acoustic signal, the acoustic signal being representative of a respiratory activity of an individual, the respiratory activity being defined by respiratory cycles each comprising an inhalation phase and an exhalation phase, the processing comprising:
detecting pause intervals in the acoustic signal; and
determining time intervals corresponding to inhalation phases and time intervals corresponding to exhalation phases by comparison, for each time interval, of a duration of the pause interval which precedes said time interval with a duration of the pause interval which follows said time interval.

11. A device comprising:
at least one processor; and
at least one non-transitory computer readable medium comprising instructions stored thereon which when executed by the at least one processor configure the device to:
receive an acoustic signal as input and store the acoustic signal;
process the stored acoustic signal to determine respiratory phases in an acoustic signal representative of a respiratory activity of an individual, the respiratory activity being defined by respiratory cycles each comprising an inhalation phase and an exhalation phase, wherein the determining comprises:
detecting pause intervals in the acoustic signal; and
determining activity intervals corresponding to inhalation phases and activity intervals corresponding to exhalation phases taking account by comparison of the duration of the pause intervals.

12. The device according to claim 11, wherein the instructions configure the device to, in response to the device identifying, at an end of the detecting, at least one succession of two pause intervals which are separated in the signal by a time interval, called aberrant interval, of duration less than a predetermined duration threshold:
replace, for each detected succession, a set consisting of the two pause intervals and said aberrant interval by a new pause interval.

13. The device according to claim 11, wherein the instructions configure the device to, in response to the device identifying, at the end of the determining, at least one time interval, called indeterminate interval, meeting a predetermined inconsistency criterion:
process, for each indeterminate interval, a portion of the acoustic signal corresponding to said indeterminate interval, by using a machine learning method; and
associate said at least one indeterminate interval with an inhalation or exhalation phase depending on results of processing the portion of the acoustic signal.

14. The device according to claim 11, wherein detecting the pause intervals is based on a processing belonging to the group consisting of:
a frequency processing of the acoustic signal;
an energy processing of the acoustic signal.

15. The device according to claim 14, wherein detecting the pause intervals is based on the frequency processing of the acoustic signal, and the frequency processing comprises:
applying a high-pass anti-noise filter to said signal to remove a first predefined frequency range from said signal and obtain a filtered signal as a function of time;
segmenting said filtered signal to obtain a plurality of samples of said filtered signal and, for each sample, estimating an average power spectral density of said sample so as to obtain a frequency signal representing the centroid frequency as a function of time;
applying a sliding time window of predetermined duration to said frequency signal and, for each window:
detecting a frequency maximum of the frequency signal in said window,
determining a frequency threshold depending on the frequency maximum detected in said window;
shaping, from said frequency signal, a first transient logic signal of logic levels defined depending on the frequency thresholds determined for said frequency signal, the pause intervals being detected depending on the first transient logic signal.

16. The device according to claim 14, wherein detecting the pause intervals is based on the energy processing of the acoustic signal, wherein the energy processing comprises:
applying a high-pass anti-noise filter and a low-pass filter to remove respectively first and second predefined frequency ranges from said signal and obtain a filtered signal as a function of time;
applying a first sliding time window of predetermined duration to said filtered signal and, for each time window, estimating the acoustic intensity of said filtered signal by applying a root mean square envelope in said window, so as to transform said filtered signal into an energy signal as a function of time;
applying a second sliding time window of duration determined by autocorrelation to said energy signal and, for each window:
detecting a minimum intensity of the energy signal in said window,
determining an intensity threshold determined as a function of the minimum intensity detected in said window;
shaping, from said energy signal, a second transient logic signal of logic levels defined depending on the intensity thresholds determined for said energy signal, the pause intervals being detected depending on the second transient logic signal.

17. The device according to claim 16, wherein the instructions configure the device to shape, by processing said first and second transient logic signals by an OR-Inclusive logic function, a final logic signal of high logic levels corresponding to said detected pause intervals.

18. The device according to claim 17, wherein the instructions configure the device to, in response to the device identifying, at the end of the shaping the final logic signal, at least one succession of two pause intervals which are separated by an aberrant interval detected in said final logic signal of duration less than the predetermined duration threshold:
replace, for each succession detected in said final logic signal, a set consisting of the two pause intervals and said aberrant interval by a new pause interval.

19. The method according to claim 1, wherein the receiving comprises receiving the acoustic sound signal from a sensor, which is configured to capture sound pressure waves.

20. The method according to claim 1, which further comprises capturing the acoustic pressure waves with the sensor and generating the acoustic signal from the captured acoustic pressure waves.
